(19) United States
(12) Patent Application Publication
(10) Pub. No.: US 2018/0103310 A1
(43) Pub. Date: Apr. 12, 2018

(54) LIGHT WEIGHT HEADPHONE

(71) Applicant: MOLDEX-METRIC, INC., CULVER CITY, CA (US)

(72) Inventors: JAMES HORNSTEIN, Studio City, CA (US); Crest Turdjian, Los Angeles, CA (US)

(73) Assignee: MOLDEX-METRIC, INC., CULVER CITY, CA (US)

(21) Appl. No.: 15/287,610

(22) Filed: Oct. 6, 2016

(51) Int. Cl. H04R 1/10; A61F 11/08

(52) U.S. Cl. CPC H04R 1/1016; A61F 11/08; H04R 2420/07; H04R 1/1083; A61F 2011/085; H04R 1/1066

(57) ABSTRACT

A light weight headphone insertable into a human ear canal having a curved portion leading to the ear drum. A main body element having a passageway through the main body element. An elongated hollow stem having two ends, one end attached to the passageway in the main body, the other end terminating in a handle. A speaker located in the handle and sound transmitted from the speaker through the hollow stem and the main body to the ear canal. The hollow stem member having a portion with a wavy configuration. Rotation of the hollow stem member will fit the hollow stem member around the curved portion in the human ear canal and seat the main body deeply in the ear canal to acoustically block the ear canal from outside noise and transmit the sound from the speaker in close proximity to the ear drum.

### BACKGROUND OF THE INVENTION

This invention relates to light weight headphones (known as earphones or earbuds) for controlling sound from portable radio communications (i.e., Bluetooth, CD players, portable media players or mobile phones), to the eardrum and also, blocking sound to the eardrum from outside of the ear to maximize the clarity of the sound and minimize any damage to the eardrum. The present invention also relates generally to light weight headphones of the push-in type. Specifically, the present invention relates to a new concept for an insert light weight headphone including an insertion stem for a push-in type of light weight headphone that permits easy and deep insertion of the earplug portion of the light weight headphone with less pressure and less contact of the stem with the sidewalls of the ear canal. This light weight headphone will fit and provide comfortable and clear sound transfer to the eardrum to a larger number of individuals than prior art light weight headphones.

### PRIOR ART

Since the indention of portable devices, such as portable radio communications (i.e., Bluetooth, CD players, portable media players or mobile phones), the light weight headphones (known as earphones or earbuds) have become more popular. For the people who prefer a simple listening experience, the new ear-fitting headphones are relatively inexpensive and more comfortable comparing to full-size over-ear headphones, as they are light in weight, small in size, they do not cover the ears and are less pressure against the ears.

While there are various models of earphones available, in general they fail into two categories-either block the external noise or allow the ambient noise to seep in.

Typically, the earphones that do Dot block the ambient noise are fitted in the outer ear, but not inserted in the ear canal, and the earphones that isolate the outside noise are inserted into the ear canal (known as in-ear headphone). The earphones fitted in the outer ear generally require separate designs to accommodate the tight and left ear. Also this type of earphone uses both friction and gravity to hold the earphone in place but can be dislodged during physical activity or exercise.

The ideal would be to have the in-ear headphone inserted deeply in the eat canal to minimize external noise and to reduce the possibility of the earphone being dislodged during use. However, the further the headphone is inserted, the smaller the size of the speaker as the ear canal narrows. The smaller speaker reduces sound volume and frequency range. It would be desirable to have a larger speaker bat also to have the ear canal blocked by an earplug portion of the headphone to minimize external noise.

### OBJECT OF THE INVENTION

It is an object of the invention to provide a new and novel light weight headphone construction to block outside noise and at the same time provide excellent sound clarity to the user of the light weight headphone and be easy and quick to insert, comfortable in the ear canal and to stay in place doting physical activity.

It is still another object of the invention to provide a new and novel light weight headphone that includes a sound blocking earplug portion that may be used with either an injection molded resilient thermoplastic elastomer material such as synthetic rubber material or a viscoelastic polymeric foam type of material.

It is another object of the invention to provide a light weight headphone which may be produced in a single size and design for both right and left ear canals, but which provides the easy and quick insertion, wearer comfort and superior sound attenuation to block noisy environmental sounds for substantially the entire adult population.

Other objects and advantages of the invention will in part be obvious and will in part appear hereinafter.

### SUMMARY OF THE INVENTION

The present invention achieves the advantages described above and eliminates the deficiencies of the prior art. The present invention relates generally to a light weight headphone including a hearing protective earplug portion and is more particularly concerned with an earplug portion of the push-in type. Specifically, the present in vend on is a light weight headphone that includes an insertion stein for the push-in type of earplug portion that permits easy, quick and deep insertion of the earplug portion with less pressure and less contact of the stem with the sidewalls of the ear canal and stay in place during use. This earplug portion will fit and provide comfortable sound attenuation of outside noise to a larger number of individuals than prior art light weight headphones. The insertion stem for the earplug portion has a unique design to accomplish the desired result. At the same time the light weight headphone can include a larger speaker located at an outside position of the light weight headphone.

The earplug portion of the present invention may be composed of different materials. One such material is resilient polymer foam material such as foam rubber, polyurethane or plasticized polyvinylchloride. For example, in U.S. Pat. No. 5,188,123 there is disclosed a push-in type earplug comprising a soft, elastic foam body element having a stiff axially oriented stem by which to facilitate insertion and removal of a body element into and from the ear canal. Another type of push-in earplug includes a central shaft and extending there from at a trout end at least a single flange member. These earplugs are generally composed of an injection molded resilient elastomer material such as synthetic rubber material or natural rubber. The flange element extends rearward from the front end of the shaft member and also extends rearward from the front end and is so spaced so as to provide a free annular space between the flange and the shaft. An earplug of this type may be seen in U.S. Pat. No. 5,957,136.

A major problem generally incurred with all of these earplugs of the prior art resides in the anatomical fact that the human ear canal is quite variable in size and geometry. Substantially, all ear canal include at least one curved section so that there is not a straight path between the outside of the ear and the inner ear. This is a protective measure in the human ear to prevent a direct path to the inner ear, which could be compromised by a finger or a stick or any other foreign object. As a result, a single type of a prior art earplug has not been found capable of accommodating the broad range of human ear canals in both size and geometry. Quite often in inserting the earplugs of the prior art, the stem member cannot be manipulated to insert the front end to the proper position to maximize attenuation without incurring discomfort by touching the sidewall of the ear canal thereby irritating the ear canal during use of the earplug.

The great majority of the existing push-in types of earplugs have straight stems generally made out of relatively stiff plastic materials (for instance the earplug described in U.S. Pat. No. 5,188,123). However, to provide proper insertion, the plastic stem of this type of earplug should be flexible/bendable as the human ear canal is not straight and varies among individuals in size and shape. However, to bend a plastic stem inside the ear canal may not be ideal and can increase the pressure and may not be comfortable for the wearer either during insertion or during wear.

The present invention includes the use of a stem design that will overcome the above deficiencies since the earplug portion of the light weight headphone is inserted using a stem member with a handle portion located outside of the ear canal. This stem design is shown in U.S. Pat. No. 8,413,663. The handle portion will also accommodate a larger speaker than could be inserted into the ear canal. In order to transmit the sound from the speaker to the ear drum, the insertion stem is hollow to provide a sound transmission path from the speaker to the end of the insertion stem where the earplug portion is located to block external noise while allowing the sound to emerge at a position close to the ear drum.

In U.S. Pat. No. 3,811,437 and Reissue Pat. No. 29,487, roll down type hearing protective earplugs composed of viscoelastic polymeric foam are shown to be compressed, inserted into the ear canal and therein al lowed to expand to result in a comfortable and complete acoustic blocking of the ear canal. This type of earplug does conform to the shape of the ear canal but with current types of stem-mounted earplugs, the stem portion does not. The present invention is directed to a design for the light weight headphone that will overcome the above deficiencies since the earplug portion is inserted using a stem member with a handle portion located outside of the ear canal.

The light weight headphone of the present invention includes an earplug portion formed by a main body element having a front end. The main body element is of circular or ovoid cross section, and may be formed of either an injection molded resilient thermoplastic elastomer material, such as synthetic rubber material or a viscoelastic polymeric foam type of material or other types of acoustic foam material that have a fast or slow recovery. An elongated stem member is attached to the main body element and may be axially located in the main body and the stem member then extends outward and may terminate at an exterior position with a handle portion that includes a speaker.

The stem member has a crooked or wavy configuration as it extends outward from the main body element within the ear canal so that as the main body element of the light weight headphone is inserted into a user's ear canal the stem member may be rotated during insertion with the handle portion to fit around the typical curved portion in the human ear canal to seat the main body element to acoustically block the ear canal while at the same time spacing the stem member within the ear canal to minimize contact with the sidewalls of the ear canal. The crooked stem member may be either axially in line with the main body element or may be axially offset or eccentric along its length to increase the spacing effect during the rotation of the light weight headphone within the ear canal. The crooked stem member may include one or more portions that are angularly displaced from adjacent portions along the length of the stem member and with such angularly displaced adjacent stem portions being curved or straight within certain dimensional ranges to fit substantially the entire adult population.

In the present invention there is no need to bend the stem to insert the earplug. Just by twisting and/or pushing the handle portion of the stem member during the insertion, the user is able to find the best position for the stem to be spaced from the sidewalls in the typical curved portion of the human ear canal. The stem member within the ear canal has less contact with the walls of the ear canal (less pressure) because of the angularly displaced adjacent stem portions being curved or straight within certain dimensional ranges forming the crooked configuration. The curved or straight angularly displaced adjacent stem portions can be two or three dimensional in nature and are designed to provide a comfortable fit to a large number of the population. In addition, as indicated above, for more versatility the rear end of the stem member that carries the earplug body could be eccentric against the centerline of the main body of the earplug so as to enhance the rotational effect.

As indicated above, the handle portion of the light weight headphone of the present invention includes a larger speaker than could be located in a headphone where the speaker is located in the ear canal. The larger speaker will provide a better sound quality with more clarity and volume than a smaller speaker. In order to transmit the sound produced by the speaker located in the handle portion of the light weight headphone through the ear canal and to the ear drum, the stem uniquely is formed as a hollow stem to transmit the sound through the hollow stem to the ear drum. The light weight headphones of the prior art that have the speaker located at an outside position, such as an earbud, do not isolate the ear canal from outside noise and also do not transmit the sound to a position adjacent to the ear drum but must travel down, and be absorbed by the walls of the ear canal before foe sound reaches the ear drum. By using a large speaker located at an outside position and transmitting the sound through a hollow stem directly to the ear drum, and providing a main body element to serve as an earplug to block outside noise, the light weight headphone of the present invention provides better sound quality and volume and a better attenuation of outside noise than prior art light weight headphones.

### BRIEF DESCRIPTION OF THE DRAWING

FIG. 1 is a front view of a first embodiment of a light weight headphone in accordance with the present invention,

FIG. 2 is a side view of the first embodiment of the light weight headphone in accordance with the present invention,

FIG. 3 is a cross-sectional view taken along line 3-3 of FIG. 2, of the first embodiment of the light weight headphone in accordance with the invention,

FIG. 4 is a cross-sectional view of a second embodiment of the light weight headphone m accordance with the invention,

FIGS. 5A and 5B are cross-sectional views, of the speaker housing, stem and earplug mount of the present invention and illustrating the method of molding the speaker housing, stem and earplug mount in one piece,

FIGS. 6A through 6C are cross-sectional views, of the speaker housing, stem and earplug mount of the present invention and illustrating the method of molding the speaker housing, stem and earplug mount in several pieces and combining the pieces into the desired structure of FIG. 6C,

FIGS. 7A and 7B are cross-sectional views, of the speaker housing, stem and earplug mount of the present invention and illustrating the method of assembling the various components into the first embodiment of FIG. 3 where the speaker is attached to a sound source with wires,

FIGS. 8A and 8B are cross-sectional views, of the speaker housing, stem and earplug mount of the present invention and illustrating the method of assembling the various components into the second embodiment of FIG. 4 where the speaker is attached to a sound source by portable radio communications i.e. Bluetooth,

FIGS. 9A through 9E are cross-sectional views of a variety of shapes of earplug portions that may be used as the front main body member to block outside sound from entering the ear canal tor the light weight headphone in accordance with the invention,

FIG. 10 illustrates a typical ear canal,

FIG. 11 is an illustration of the light weight headphone in accordance with the invention using the plug portion of FIG. 9A being inserted into the ear canal and with the stem member being manipulated so that the earplug portion can be seated in the ear canal for maximum attenuation while at the same time being rotated to ensure clearance between the stem member and the sidewalls of the ear canal,

FIG. 12 is an illustration of the light weight headphone in accordance with the invention using the plug portion of FIG. 9D being inserted into the ear canal and with the stem member being manipulated by being pushed in so that the earplug can be sealed in the ear canal for maximum attenuation while at the same time the stem member rotated to ensure clearance between the stem member and the sidewalls of the ear canal.

### DETAILED DESCRIPTION OF THE INVENTION

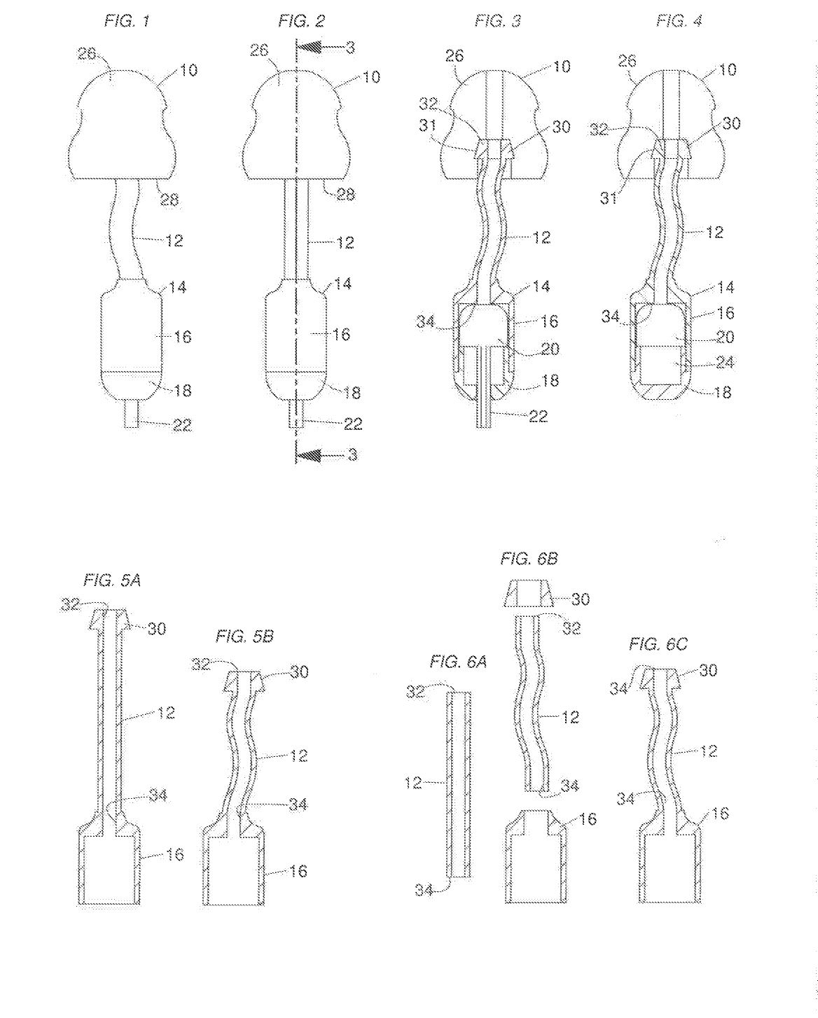

Referring now to FIGS. 1 through 4, two embodiments of the present invention are shown and the same reference numerals refer to the structures having a similar function including a main body element 10 and an elongated stem member 12 extending outward and axially from the and at least partially within the main body element or attached to the main body element 10. The stem 12 terminates in a handle 14 that serves as an enclosure for a sneaker. The handle 14 is formed in two pieces, a speaker housing 16 and a housing cap 18. As can be seen in the two cross-sectional views, FIGS. 3 and 4, the handle 14 receives a speaker 20. In FIG. 3, the speaker 20 is connected to a source of sound using a wire 22 in a conventional manner. In FIG. 4, the speaker 20 is connected to a source of sound using a radio receiver 24 in a conventional manner such as through a Bluetooth transmission.

In FIGS. 1 through 4, the earplug portion or main body element 10 of the invention uses a soft, resilient polymeric foam body each comprising a smoothly contoured, homogenous, viscoelastic main body element adapted in size and shape to be inserted into the human ear canal. The main body element 10 can take a variety forms and can be constructed of various materials other than the resilient polymeric foam. In particular, the main body element can have a hemispheric pod shape as shown in FIGS. 1 through 4 and FIG. 9A. Also the main body element cars have a bullet shape as shown in FIG. 9D or a straight sided shape as shown in FIG. 9C. These shapes would typically be constructed of the resilient polymeric foam.

The main body element 10 may also have other shapes such as lateral projections in the nature of one or more flange elements, as shown in FIG. 9C, or an elongated bulbous curved shape with a flange portion, as shown in FIG. 9E. These shapes would typically be constructed of an injection molded resilient elastomer material such as synthetic or natural rubber material.

All of the earplug portions or main body elements 10 are similar in nature since they all include a front end 26 and a base end 28. While the preferred cross sectional shape of the main body element 10 is circular, it will be appreciated that the cross sectional shape may also be ovoid or elliptical.

The stem member 12 is an elongated member that is tubular or hollow throughout its cross section and may he uniform or non-uniform along its length. In a preferred structure, the elongated stem has two ends, one end 32 is located in and secured by a tapered knob 30 by an interference fit in a conforming opening 31 in the interior of the front end portion 26 of the main body element 10. The stem 12 then extends axially and outward from the front-end portion 26 and through the base end 28. The other end 34 of the stem may terminate at a point exterior to or adjacent to the base end 28 and forms the handle portion 14 of the stem 12. The stem is sufficiently stiff so as to allow manipulation of the main body element 10 so that the light weight headphone can be seated in the ear canal for maximum attenuation while at the same time being rotated to ensure clearance between the stem member and the sidewalls of the ear canal.

As can be seen in FIGS. 3 and 4, the hollow stem 12 and, in particular, the portion of the hollow stem between the one end 32 and the handle portion 14 has a crooked or wavy configuration and specifically has a curved adjacent hollow stem portion that would conform to the shape of a typical ear canal as it extends inward from the outside to the inner ear. Since there can be variations in the shape of the ear canal between different users of earplugs, the rotation of the earplug as it is being inserted into the ear canal creates the ability to accommodate these variations in the shape of the ear canal by changing the position of the curved adjacent hollow stem portions of the stem 12 in the ear canal.

The cross sectional dimension of the hollow stem 12 should not be so great as to prevent easy insertion of the earplug portion into ear canals of small size. The hollow stem 12 can be of any convenient length such that, for the embodiments of FIGS. 9A and 9B, the handle portion 14 extends beyond the main body element 10. The handle portion 14 is formed as an enlarged end to not only provide a housing for the speaker 20 but also provide an insertion and removal member for the main body element 10. The exact length selected for the hollow stem 12 including the handle 14 will largely be a matter of choice but the dimensions of the portion of the hollow stem between the one end 32 and the handle portion 14 will have a range in length and deviation from a center axis that will accommodate the largest number of variations in size and shape of ear canals. For the embodiments of FIGS. 9D and 9E, the handle portion 14 is located adjacent to the base end 28 of the main body element 10 but the same dimensional considerations for the hollow stem 12 as to length and deviation from a center axis that will accommodate the largest number of variations in size and shape of ear canals will apply.

The crooked stem member may be either axially in line with the main body or may be axially offset or eccentric along its length to increase the spacing effect during the rotation of the earplug portion within the ear canal. The dimensions, geometry and material of construction employed for the hollow stem 12 are selected so that the hollow stem 12 is sufficiently stiff to allow manipulation of the main body element 10 so that the earplug portion can be seated in the ear canal for maximum attenuation while at the same time being rotated to ensure clearance between the hollow stem member and the sidewalls of the ear canal. For additional details of the design of the hollow stem, reference is made to U.S. Pat. No, 8,413,663 assigned to the same assignee as the instant application.

The contoured main body element 10 that extends from the front end 26 may be formed with a rounded configuration to aid insertion of the earplug portion. The length of the main body element 10 relative to its maximum cross sectional dimension is subject to considerable variation as can be seen in the various embodiments of the invention and is not normally critical. For example, the shape of main body element 10 of the earplug shown in FIGS. 9A and 9B each include a hemispheric pod portion and their length to diameter ratio contrasts with the embodiments of FIGS. 9C through 9E, where the body elements 10 are elongated and have a larger length to diameter ratio.

FIGS. 5A and 5B illustrate forming the hollow stem 12, the speaker housing 16 and the tapered knob 30 as a single molded one piece member. As can be seen in FIG. 5A, in the initial mold, the one piece member is formed with a straight hollow stem. As can be seen in FIG. 5B, the hollow stem 12 is then further molded into the desired configuration described above to accommodate these variations in the shape of the ear canal by changing the position of the curved adjacent hollow stem portions of the stem 12 in the ear canal.

FIGS. 6A through 6C illustrate forming the hollow stem 12, the speaker housing 16 and the tapered knob 30 as separate molded individual members. As can be seen in FIG. 6A, in the initial mold the hollow stem member id formed with a straight hollow stem. As can be seen in FIG. 6B, the hollow stem 12 is then further molded into the desired configuration described above to accommodate these variations in the shape of the ear canal by changing the position of the curved adjacent hollow stem portions of the stem 12 in the ear canal. Also, the speaker housing 16 and the tapered knob 30 are molded as individual members. Finally as seen in FIG. 6C, the individual members are assembled and joined together by any appropriate method such as ultrasonic welding or adhesive bonding.

FIGS. 7A and 7B illustrate the assembly of the speaker 20 in the handle portion 14 when the speaker is connected to a remote source of sound using wires 22. As can be seen in FIG. 7A, the wires 22 are inserted through the housing cap 18 into the interior of the housing cap 18 to be attached to the speaker 20 as shown in FIG. 7B. The assembly shown in FIG. 7B can then be positioned within the speaker housing 16 to form the complete light weight headphone as shown in FIG. 3 that uses a wired connection to the remote source of sound. Also, as seen in FIG. 3, the light weight headphone is completed using an earplug portion 10 that is held in place using the tapered knob 30 and the corresponding recess 31 in the earplug portion 10. Use structure of the tapered knob 30 and recess 31 allows the earplug portion 10 to be removed and replaced by a fresh earplug portion when soiled. It is to be appreciated that other appropriate interference fit structures can he used in place of the tapered knob 30 and recess 31.

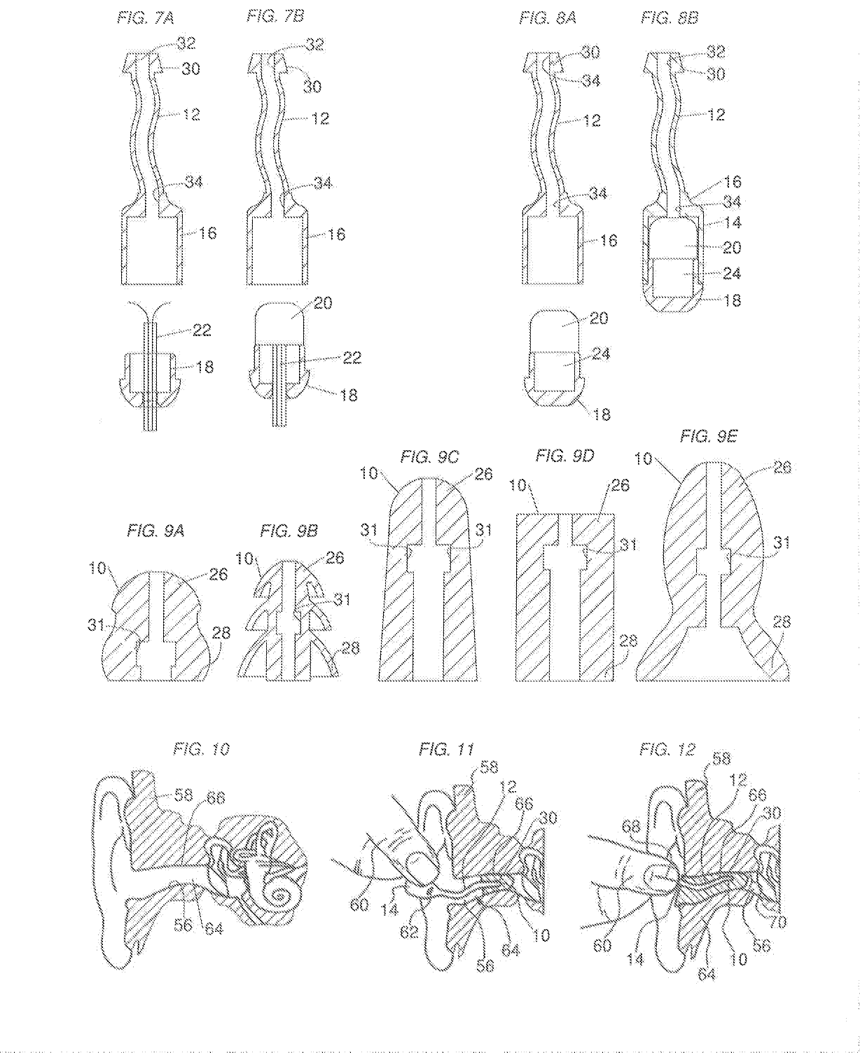

FIGS. 8A and 8B illustrate the assembly of the speaker 20 in the handle portion 14 when the speaker is connected to a remote source of sound using a radio receiver 24. As can be seen in FIG. 8A, the interconnected speaker 22 and radio receiver 24 are positioned in the housing cap 18. The assembly of the speaker 22, radio receiver 24 and housing cap 18 can then be positioned within the speaker housing 16 to form the complete light weight headphone as shown in FIG. 8B and FIG. 4 that use the radio connection to the remote source of sound. Also, as seen in FIG. 4, the light weight headphone is completed using an earplug portion 10 that is held in place using the tapered knob 30 and the corresponding recess 31 in the earplug portion 10. The structure of the tapered knob 30 and recess 31 allows the earplug portion 10 to he removed and replaced by a fresh earplug portion when soiled. It is to be appreciated that other appropriate interference fit structures can be used in place of the tapered knob 30 and recess 31.

FIG. 10 illustrates a typical ear structure. In FIG. 10 it can be seen that the typical ear structure includes the curved structure of the external auditory meatus or ear canal with sidewalls leading to the eardrum. In appearance, it is a slightly curved tube that extends inward from the floor of the auricle, or protruding portion of the outer ear, and ends blindly at the eardrum membrane, which separates it from foe middle ear.

FIG. 11 illustrates the earplug portions or main body element 10 of the earplug and in particular represents the embodiments of FIGS. 3 and 4 using earplug portions 10 shown in FIGS. 9A and 9B being inserted into an ear canal 56 of a human subject 58 and with the stem member 12 being manipulated by the fingers 60 of the human subject 58 so that the earplug portion can be seated in the ear canal 56 for maximum attenuation while tit the same time being rotated as shown by arrow 62 to ensure clearance to minimize contact between the stem member 12 and sidewalls 64 of the typical curved portion 66 in the human ear canal 56. If the crooked stem member 12 is axially offset or eccentric along its length, this increases the spacing effect during the rotation of the stem member within the ear canal. This structure acoustically blocks the ear canal from outside noise and transmits the sound from the speaker in close proximity to the ear drum.

FIG. 12 illustrates the earplug portion or main body element 10 and in particular represents the embodiments of FIGS. 9D and 9E being inserted into an ear canal 56 of a human subject 58 and with the handle portion 36 and thereby the stem member 12 being pushed in as shown by arrow 68 by the fingers 60 of the human subject 58 so that the earplug can be seated to fit around the typical curved portion in the human ear canal 56 for maximum attenuation while at the same time the stem member 12 rotates as shown by arrow 70 to ensure clearance between the stem member 12 and sidewalls 64 of the ear canal 56. The rotation of the stem member 12 occurs during insertion since the stem member adjusts to the shape of the ear canal to find the path of least resistance. If the crooked stem member is axially offset or eccentric along its length, this increases the spacing effect during the rotation of the stem member within the ear canal. This structure acoustically blocks the ear canal from outside noise and transmits the sound from the speaker in close proximity to the ear drum.

The present invention is not to be limited to any specific design for the main body element or stem member including the handle portion of the stem member. There are a large number of designs currently used for a main body element of an earning and the only limiting requirement for the present invention is that the main body element be capable of being inserted a significant distance into the ear canal to provide high attenuation levels. Similarly, the handle portion of the stem member can take any form to contain a speaker and also can be manipulated by a user of the earplug. Therefore, the handle portion can be round, oval, straight or bent or any other shape and can be an extension of the stem itself.

Several human subjects, having varying ear canal sizes and geometries, have used the light weight headphone of the present invention in noisy environments. These subjects have reported that the light weight headphone was easy to use, was comfortable throughout insertion, wearing and removal and provided adequate attenuation for their needs and acoustically blocks the ear canal from outside noise and transmits the sound from the speaker in close proximity to the ear drum.

The present invention has been described wherein outside noise is acoustically blocked but if it is desirable to allow in some outside sound for safety or any other purpose, this can be accomplished by providing a small opening in the hollow stem near the handle. Alternately, the earphone of the present invention can also include a mechanism so that the earphone can be switched between a first position where outside noise is substantially blocked and a second position where desirable outside noise can enter the ear canal. This could be accomplished by the use of a removable plug for the small opening described above. Alternatively, a more elaborate structure could be used as shown in U.S. Pat. No. 9,282,390. As a further example of a structure that allows some outside sound to reach the eardrum, the main body member could include an additional passageway not connected to the hollow stem, so that outside noise can pass to the ear drum. This could take the form of an actual opening passing through the main body member or recesses on the side of the main body member so that the main body member does not completely seal the ear canal.

Although the invention has been described and shown using specific preferred embodiments, it is to be appreciated that various adaptations and modifications may be made and the invention is only to be limited by the appended claims.

1. A light weight headphone insertable into a human ear canal having sidewalls and a typical curved portion leading to the ear drum, comprising, a soft main resilient body having a main body element having a front end and an exterior shape and a passageway extending through the main body element, an elongated hollow stem having two ends, one end attached to the passageway through the main body element, the hollow stem extending outward from the main body element and away from the front end, the other end of the hollow stem terminating in a handle portion at the end of the hollow stem, a speaker for transmitting sound from a remote source, the handle portion forming a speaker housing for housing the speaker in communication with the hollow stem for transmitting sound from the speaker through the hollow stem and the passageway in the main body element to the ear canal, the hollow stem being sufficiently stiff as to allow manipulation of the main body element during insertion into the ear canal, the hollow stem member having a portion with a crooked or wavy configuration as it extends outward from the main body to the handle portion and so that during insertion of the front end of the main body element into the ear canal, rotation of the hollo w stem member with the handle portion will fit the hollow stem member around the typical curved portion in the human ear canal so as to seat the main body element deeply in the ear canal to transmit the sound from the speaker in close proximity to the ear drum while at the same time spacing the hollow stem member within the typical curved portion of the ear canal to minimize contact with the sidewalls of the ear canal.

2. The light weight headphone of claim 1 wherein the exterior shape of the main body element is bullet shaped.

3. The light weight headphone of claim 1 wherein the exterior shape of the main body element is a hemispherical pod shape.

4. The light weight headphone of claim 1 wherein the exterior shape of the main body element has lateral projections in the nature of one or more flange elements.

5. The earplug of claim 1 wherein the exterior shape of the main body element is elongated to enclose a substantial portion of the length of the stem member.

6. The light weight headphone of claim 1 wherein the exterior shape of the main body element is elongated to enclose a substantial portion of the length of the stem member.

7. The light weight headphone of claim 1 wherein the exterior shape of the main body element is elongated to enclose a substantial portion of the length of the stem member and wherein the main body element includes a flared shape adjacent the handle portion of the stem member.

8. The light weight headphone of claim 1 wherein the main body element is formed of a resilient foam material.

9. The light weight headphone of claim 1 wherein the main body element is an injection molded resilient thermoplastic elastomer material such as synthetic rubber material.

10. The light weight headphone of claim 1 wherein the handle portion of the stem and the main body element are located along the same axis.

11. The light weight headphone of claim 1 wherein the handle portion of the stem is offset from the main body element and the handle portion of the stem and the main body element are located along two separate axes.

12. The light weight headphone of claim 1 wherein the stem member having the crooked or wavy configuration as it extends outward from the main body and within the ear canal is formed into a curved shape and wherein the rotation of the stem as it is being inserted into the ear canal accommodates variations in the shape of the ear canal by changing the position of the curved shape portion of the stem in the ear canal.

13. The light weight headphone of claim 1 wherein the handle portion of the stem is formed as an enlarged portion of the stem.

14. The light weight headphone of claim 1 wherein the handle portion of the stem is formed as an enlarged portion of the stem and in two pieces, one piece formed as the speaker housing to receive and house the speaker and another piece formed as a housing cap to close off the end of the speaker housing.

15. The light weight headphone of claim 1 wherein the speaker for transmitting sound from a remote source is connected to the remote source using wires passing through the handle portion of the stem.

16. The light weight headphone of claim 1 wherein the speaker for transmitting sound from a remote source is connected to the remote source using a radio receiver located within the handle portion of the stem.
